(19) United States
(12) Patent Application Publication
(10) Pub. No.: US 2004/0132706 A1
(43) Pub. Date: Jul. 8, 2004

(54) Composition comprising a catalyst for the dismutation of superoxide and use of the composition for preventing and treating hypotension

(76) Inventor: Daniela Salvemini, Chesterfield, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 10/398,705

(22) PCT Filed: Oct. 5, 2001
(86) PCT No.: PCT/US01/42502

(51) Int. Cl. A61K 31/555

(52) U.S. Cl. 514/184; 540/465

(57) ABSTRACT

The present invention relates to pharmaceutical and veterinary compositions and methods using such compositions for the treatment of hypotension. Such compositions contain a catalyst for the dismutation of superoxide, including superoxide dismutase enzyme (SOD) and small molecular weight organic ligand mimics of that enzyme (SOD mimetics or SODms) which may be administered alone or in combination with a catecholamine pressor agent. Applications described include treatments for hypotension resulting from septic, cardiogenic, hypovolemic, anaphylactic or burn-induced shock treatments.

### FIELD OF INVENTION

This invention relates to methods of preventing and treating hypotension in a mammal resulting from e.g., septic, cardiogenic, anaphylactic or burn-induced shock by administering therapeutic amounts of catalysts for the dismutation of superoxide to the mammal. Also provided are pharmaceutical compositions comprising catalysts for the dismutation of superoxide for use in these methods.

### BACKGROUND OF THE INVENTION

Hypotension is a hemodynamic condition characterized by low blood pressure resulting from reduced vascular resistance despite increased levels of endogenous catecholamines. This condition persists despite the maintenance of normal blood volume (normovolemia). Another characteristic of this condition is hyporeactivity, the loss of vascular responses, which develops to both exogenous and presumably, endogenous catecholamines. Hypotension often develops in cases of septic shock, cardiogenic shock, hypovolemic shock, anaphylactic shock and burn-induced shock. The presence and persistence of hypotension in these patients has been correlated with a decreased survival rate, and is considered to be one of the life-threatening conditions associated with these shock states.

One characteristic of these shock states such as sepsis, is the large increase in the production of free radicals, including superoxide anions (O₂⁻) within the body. See Ischiropoulos et al., Arch. Biochem. Biophys. 298: 446-451 (1992); Taylor et al., Arch. Biochem. Biophys., 316: 70-76 (1995). Superoxide anions are normally removed in biological systems by the formation of hydrogen peroxide and oxygen in the following reaction (hereinafter referred to as dismutation):

O₂⁻+O₂⁻+2H⁺→O₂+H₂O₂

This reaction is catalyzed in vivo by the ubiquitous superoxide dismutase enzyme.

Several non-proteinaceous catalysts which mimic this superoxide dismutating activity have been discovered. A particularly effective family of non-proteinaceous catalysts for the dismutation of superoxide consists of the manganese(II), manganese(III), iron(II) or iron(III) complexes of nitrogen-containing fifteen-membered macrocyclic ligands which catalyze the conversion of superoxide into oxygen and hydrogen peroxide, as described in U.S. Pat. Nos. 5,874,421 and 5,637,578, all of which are incorporated herein by reference. See also, Weiss, R. H., et al., “Manganese(II)-Based Superoxide Dismutase Mimetics: Rational Drug Design of Artificial Enzymes”, Drugs of the Future 21: 383-389 (1996); and Riley, D. P., et al., “Rational Design of Synthetic Enzymes and Their Potential Utility as Human Pharmaceuticals” (1997) in CatTech, I, 41. These mimics of superoxide dismutase have been shown to have a variety of therapeutic effects, including anti-inflammatory activity. See Weiss, R. H., et al., “Therapeutic Aspects of Manganese (II)-Based Superoxide Dismutase Mimics” In “Inorganic Chemistry in Medicine”, (Farrell, N., Ed.), Royal Society of Chemistry, in Press; Weiss, R. H., et al., “Manganese-Based Superoxide Dismutase Mimics: Design, Discovery and Pharmacologic Efficacies” (1995), In “The Oxygen Paradox” (Davies, K. J. A., and Ursini, F., Eds.) pp. 641-651, CLEUP University Press, Padova, Italy; Weiss, R. H., et al., J. Biol. Chem., 271: 26149 (1996); and Hardy, M. M., et al., J. Biol. Chem. 269: 18535-18540 (1994). Other non-proteinaceous catalysts which have been shown to have superoxide dismutating activity are the salen-transition metal cation complexes, as described in U.S. Pat. No. 5,696,109 and complexes of porphyrins with iron and manganese cations.

Current clinical therapy for hypotension includes fluid resuscitation therapy coupled with intravenous infusions of the catecholamines norepinephrine (NE) and dopamine. However, this clinical therapy is limited as a result of hyporeactivity of the vascular system to the catecholamine infusion. Despite repeated catecholamine doses, maintenance of an acceptable blood pressure (usually >90 mmHg) is often unattainable. Although non-catecholamine pressor agents, such as vasopressin, are being developed, they often have undesirable side effects and are difficult to produce. See U.S. Pat. No. 5,990,273.

Thus, the need presently exists for compositions and methods for preventing and treating hypotension in mammals suffering from various shock states by preventing the decrease of mean arterial pressure. Furthermore, a need exists for pharmaceutical compositions which prevent and reverse the continued decrease of mean arterial pressure associated with hypotension.

### SUMMARY OF THE INVENTION

Accordingly, an object of the present invention is to provide pharmaceutical and veterinary compounds and methods which inhibit the continued fall in mean arterial pressure associated with hypotension such as that resulting from various shock states e.g., septic shock and anaphylactic shock. Applicants have discovered that treatment with catalysts for the dismutation of superoxide, including superoxide dismutase enzyme (SOD) and small molecular weight organic ligand mimics of that enzyme (SOD mimetics or SODms) results in preventing in vitro deactivation of catecholamines. Moreover, this deactivation appears to account for the hyporeactivity to exogenous catecholamines observed in cases of hypotension, thus suggesting that the deactivation of endogenous norepinephrine by superoxide may contribute significantly to this aspect of the vascular crisis.

One aspect of the invention is to provide compounds and methods to treat hypotension by removing superoxide, thus protecting exogeneous and endogeneous catecholamines from autooxidation. In doing so, treatment of shock states which are currently difficult as a result of the toxic side effect of hypotension can take place. Accordingly, by preventing or limiting the deactivation of catecholamines, hyporeactivity and hypotension are reversed, and chances of survival are improved.

Another aspect of the present invention is to provide methods of inhibiting a fall in mean arterial blood pressure in a mammal, preferably a human, by administering to the mammal a mean arterial pressure sustaining amount of a catalyst for the dismutation of superoxide.

Yet another aspect of the present invention is to provide a method for increasing mean arterial pressure in a mammal suffering from hypotension which comprises administering to the mammal a mean arterial pressure increasing amount of a catecholamine pressor agent and a catalyst for the dismutation of superoxide. Relatedly, pharmaceutical compositions are provided which comprise catecholamine pressor agents, catalysts for the dismutation of superoxide and a pharmaceutically acceptable carrier. When administered to a mammal with hypotension, these pharmaceutical compositions inhibit the degradation of the catecholamines, allowing the catecholamine pressor agent to improve vascular tone and to increase the mean arterial blood pressure of the mammal.

A further aspect of the present invention is to provide methods of treatment or prophylaxis of various shock states such as septic shock, cardiogenic shock, hypovolemic shock, anaphylactic shock and burn-induced shock by inhibiting or treating hypotension, the method comprising administering a mean arterial pressure sustaining amount of a catalyst for the dismutation of superoxide.

Other objects and features will be in part apparent and in part pointed out hereinafter.

### BRIEF DESCRIPTION OF THE DRAWINGS

FIG. 1A is a graph of the chemical detection, as measured by HPLC, of norepinephrine (open bars) or epinephrine (solid bars) which is reduced after incubation with hypoxanthine/xanthine oxidase (HX/XO). This reduction is prevented in the presence of the SOD mimetic M40403 (10⁻⁶ M; n=6; *P<0.05; P<0.05). The incubation period was 5 min.

FIG. 1B is a graph indicating that the ability of norepinephrine (0.5 g/Kg), given as a bolus i.v. injection to an anaesthetized rat, to increase MAP (open bars) is prevented after incubation with HX/XO (solid bars). This ability to increase MAP is preserved with the inclusion of M40403 in the incubate (hatched bars; n=6; *P<0.05). The incubation period was 5 min.

FIG. 2 is a graphic representation of the increase in MAP of the anaesthetized rat by the administration of norepinephrine (0.1-1 g/Kg; ▪). LPS (4 mg/Kg) administered to the anesthetised rat results in the development of hyporeactivity to norepinephrine (0.1-1 g/Kg) within 1 h (▴). This hyporeactivity is reversed by administration of M40403 (1 mg/Kg) to the LPS treated rat (⋄). The reactivity to norepinephrine in saline treated rats is not affected by M40403. (●; n=6 for all).

FIG. 3 is a graphic representation of the development of irreversible hypotension in the anaesthetized rat (□; n≈10) resulting from the administration of LPS (4 mg/Kg i.v). Treatment with M40403 (0.25 mg/Kg/h) at 1 h post LPS prevents this fall in MAP (Δ; n≈10). M40403 (0.25 mg/Kg) administered at 5 h post LPS reverses the fall in MAP (▴; n≈10). Control animals are represented by ♦.

FIGS. 4A and 4B are graphs which demonstrate that plasma concentrations of epinephrine and norepinephrine increase over time after administration of LPS (4 mg/Kg, i.v; open bars). In rats treated with LPS and M40403 (0.25 mg/Kg/h given at 1 h post LPS; solid bars) the plasma concentrations of the catecholamines are significantly higher (n≈10 for all; *P<0.05). In these experiments there were no surviving control rats left alive for a 9 h measurement (n=10 for all; *P<0.05).

FIG. 4C is a graph which demonstrates that plasma adrenochrome concentrations increase over time after administration of LPS (4 mg/Kg, i.v; open bars). In rats treated with LPS and M40403 (0.25 mg/Kg/h given at 1 h post LPS; solid bars) the plasma concentrations of adrenochromes are significantly lower. In these experiments there were no surviving control rats left alive for a 9 h measurement (n=10 for all; *P<0.05).

FIG. 5 is a graph indicating that administration of norepinephrine (1 μg/Kg, bolus i.v. injection) to an anaesthetized rat increases MAP (open bars) and is prevented by administration of LPS (solid bars). In rats treated with FeTMPS (15 mg/kg given at 1 h post LPS; hatched bars) hyporeactivity to exogeneous NE is prevented.

FIG. 6 is a graphic representation of the increase in MAP of the anaesthetized rat by the administration of norepinephrine (0.1-1 g/Kg; ▪). LPS (4 mg/Kg) administered to the anesthetised rat results in the development of hyporeactivity to norepinephrine (0.1-1 g/Kg)(□). This hyporeactivity is reversed by administration of FeTMPS (15 mg/Kg) to the LPS treated rat (⋄). The reactivity to norepinephrine in saline treated rats is not significantly affected by FeTMPS. (▴; n=6 for all).

FIG. 7 is a graphic representation of the therapeutic treatment with FeTMPS to an anesthetized rat. Administration of administration of LPS (4 mg/Kg i.v) resulted in the development of irreversible hypotension (□). Therapeutic treatment with FeTMPS (10 mg/Kg/h) at 1 h post LPS prevents this fall in MAP (Δ). Control animals are represented by FIG. 8 is a graphic representation of the prophylactic treatment with FeTMPS to an anesthetized rat. Administration of administration of LPS (4 mg/Kg i.v) resulted in the development of irreversible hypotension(□). Prophylactic treatment with FeTMPS (10 mg/Kg/h) at 1 h post LPS prevents this fall in MAP (Δ). Control animals are represented by ♦.

### DEFINITIONS AND ABBREVIATIONS

To facilitate understanding of the invention, a number of terms as used herein are defined below:

The term “hypotension” means a hemodynamic condition characterized by raised blood pressure which persists despite the maintenance of normal blood volume (normovolemia). Generally, a patient or animal is suffering from hypotension when the mean arterial pressure is less than 90 mmHg for at least one hour despite adequate ventricular filling pressures (pulmonary artery wedge pressure [PAWP] of at least 12 mmHg) or despite a sufficient central venous pressure (CVP) of at least 8 mmHg. Other indicators of hypotension are the failure of the hypotensive state to respond to aggressive initial fluid therapy (such as the administration of 500 ml of isotonic crystalloid, 25 gm or albumin, or 200 ml of other colloids (e.g. hydroxyethyl starch)) or the need for pressor doses of dopamine (>5 g/kg/min), norepinephrine or other pressor agents to maintain a systolic blood pressure of 90 mmHg.

As used herein, “NE” refers to norepinephrine. It should be noted that NE is commonly referred to in the art as noradrenaline (NA) and that epinephrine is commonly referred to in the art as adrenaline.

As used herein, “MAP” refers to mean arterial pressure.

The term “mean arterial pressure sustaining amount”, as used in this application, means the amount of a compound needed to maintain the mean arterial pressure of a mammal suffering from or at imminent risk of suffering from hypotension associated with various shock states in the normotensive range, preferably from about 70 to about 130 mmHg for at least 30 minutes.

The term “mean arterial pressure increasing amount”, as used in this application, means the amount of a compound needed to increase the mean arterial pressure of a mammal suffering from hypotension associated with various shock states from its hypotensive state to the normotensive range, preferably from about 70 to about 130 mmHg for at least 30 minutes.

The term “non-proteinaceous catalysts for the dismutation of superoxide” means a low-molecular weight catalyst for the conversion of superoxide anions into hydrogen peroxide and molecular oxygen. These catalysts commonly consist of an organic ligand and a chelated transition metal ion, preferably manganese(II), manganese(III), iron(II) or iron(III). The term may include catalysts containing short-chain polypeptides (under 15 amino acids) or macrocyclic structures derived from amino acids, as the organic ligand. The term explicitly excludes a superoxide dismutase enzyme obtained from any species.

The term “substituted” means that the described moiety has one or more substituents comprising at least 1 carbon or heteroatom, and further comprising 0 to 22 carbon atoms, more preferably from 1 to 15 carbon atoms, and comprising 0 to 22 heteroatoms, more preferably from 0 to 15 heteroatoms. As used herein, “heteroatom” refers to those atoms that are neither carbon nor hydrogen bound to carbon and are selected from the group consisting of: O, S, N, P, Si, B, F, Cl, Br, or I. These atoms may be arranged in a number of configurations, creating substituent groups which are unsaturated, saturated, or aromatic. Examples of such substituents include branched or unbranched alkyl, alkenyl, or alkynyl, cyclic, heterocyclic, aryl, heteroaryl, allyl, polycycloalkyl, polycycloaryl, polycycloheteroaryl, imines, aminoalkyl, hydroxyalkyl, hydroxyl, phenol, amine oxides, thioalkyl, carboalkoxyalkyl, carboxylic acids and their derivatives, keto, ether, aldehyde, amine, amide, nitrile, halo, thiol, sulfoxide, sulfone, sulfonic acid, sulfide, disulfide, phosphonic acid, phosphinic acid, acrylic acid, sulphonamides, amino acids, peptides, proteins, carbohydrates, nucleic acids, fatty acids, lipids, nitro, hydroxylamines, hydroxamic acids, thiocarbonyls, thiocarbonyls, borates, boranes, boraza, silyl, silaza, siloxy, and combinations thereof.

The term “alkyl”, alone or in combination, means a straight-chain or branched-chain alkyl radical containing from 1 to about 22 carbon atoms, preferably from about 1 to about 18 carbon atoms, and most preferably from about 1 to about 12 carbon atoms. Examples of such radicals include, but are not limited to, methyl, ethyl, n-propyl, isopropyl, n-butyl, isobutyl, sec-butyl, tert-butyl, pentyl, iso-amyl, hexyl, octyl, nonyl, decyl, dodecyl, tetradecyl, hexadecyl, octadecyl and eicosyl.

The term “alkenyl”, alone or in combination, means an alkyl radical having one or more double bonds. Examples of such alkenyl radicals include, but are not limited to, ethenyl, propenyl, 1-butenyl, cis-2-butenyl, trans-2-butenyl, iso-butylenyl, cis-2-pentenyl, trans-2-pentenyl, 3-methyl-1-butenyl, 2,3-dimethyl-2-butenyl, 1-pentenyl, 1-hexenyl, 1-octenyl, decenyl, dodecenyl, tetradecenyl, hexadecenyl, cis- and trans-9-octadecenyl, 1,3-pentadienyl, 2,4-pentadienyl, 2,3-pentadienyl, 1,3-hexadienyl, 2,4-hexadienyl, 5,8,11,14-eicosatetraenyl, and 9,12,15-octadecatrienyl.

The term “alkynyl”, alone or in combination, means an alkyl radical having one or more triple bonds. Examples of such alkynyl groups include, but are not limited to, ethynyl, propynyl (propargyl), 1-butynyl, 1-octynyl, 9-octadecynyl, 1,3-pentadiynyl, 2,4-pentadiynyl, 1,3-hexadiynyl, and 2,4-hexadiynyl.

The term “cycloalkyl”, alone or in combination means a cycloalkyl radical containing from 3 to about 10, preferably from 3 to about 8, and most preferably from 3 to about 6, carbon atoms. Examples of such cycloalkyl radicals include, but are not limited to, cyclopropyl, cyclobutyl, cyclopentyl, cyclohexyl, cycloheptyl, cyclooctyl, and perhydronaphthyl.

The term “cycloalkylalkyl” means an alkyl radical as defined above which is substituted by a cycloalkyl radical as defined above. Examples of cycloalkylalkyl radicals include, but are not limited to, cyclohexylmethyl, cyclopentylmethyl, (4-isopropylcyclohexyl)methyl, (4-t-butyl-cyclohexyl)methyl, 3-cyclohexylpropyl, 2-cyclohexylmethylpentyl,3-cyclopentylmethylhexyl, 1-(4-neopentylcyclohexyl)methylhexyl, and 1-(4-isopropylcyclohexyl)methylheptyl.

The term “cycloalkylcycloalkyl” means a cycloalkyl radical as defined above which is substituted by another cycloalkyl radical as defined above. Examples of cycloalkylcycloalkyl radicals include, but are not limited to, cyclohexylcyclopentyl and cyclohexylcyclohexyl.

The term “cycloalkenyl”, alone or in combination, means a cycloalkyl radical having one or more double bonds. Examples of cycloalkenyl radicals include, but are not limited to, cyclopentenyl, cyclohexenyl, cyclooctenyl, cyclopentadienyl, cyclohexadienyl and cyclooctadienyl.

The term “cycloalkenylalkyl” means an alkyl radical as defined above which is substituted by a cycloalkenyl radical as defined above. Examples of cycloalkenylalkyl radicals include, but are not limited to, 2-cyclohexen-1-ylmethyl, 1-cyclopenten-1-ylmethyl, 2-(1-cyclohexen-1-yl)ethyl, 3-(1-cyclopenten-1-yl)propyl, 1-(1-cyclohexen-1-ylmethyl)pentyl, 1-(1-cyclopenten-1-yl)hexyl, 6-(1-cyclohexen-1-yl)hexyl, 1-(1-cyclopenten-1-yl)nonyl and 1-(1-cyclohexen-1-yl)nonyl.

The terms “alkylcycloalcyl” and “alkenylcycloalkyl” mean a cycloalkyl radical as defined above which is substituted by an alkyl or alkenyl radical as defined above. Examples of alkylcycloalkyl and alkenylcycloalkyl radicals include, but are not limited to, 2-ethylcyclobutyl, 1-methylcyclopentyl, 1-hexylcyclopentyl, 1-methylcyclohexyl, 1-(9-octadecenyl)cyclopentyl and 1-(9-octadecenyl)cyclohexyl.

The terms “alkylcycloalkenyl” and “alkenylcycloalkenyl” means a cycloalkenyl radical as defined above which is substituted by an alkyl or alkenyl radical as defined above. Examples of alkylcycloalkenyl and alkenylcycloalkenyl radicals include, but are not limited to, 1-methyl-2-cyclopentyl, 1-hexyl-2-cyclopentenyl, 1-ethyl-2-cyclohexenyl, 1-butyl-2-cyclohexenyl, 1-(9-octadecenyl)-2-cyclohexenyl and 1-(2-pentenyl)-2-cyclohexenyl.

The term “aryl”, alone or in combination, means a phenyl or naphthyl radical which optionally carries one or more substituents selected from alkyl, cycloalkyl, cycloalkenyl, aryl, heterocycle, alkoxyaryl, alkaryl, alkoxy, halogen, hydroxy, amine, cyano, nitro, alkylthio, phenoxy, ether, trifluoromethyl and the like, such as phenyl, p-tolyl, 4-methoxyphenyl, 4-(tert-butoxy)phenyl, 4-fluorophenyl, 4-chlorophenyl, 4-hydroxyphenyl, 1-naphthyl, 2-naphthyl, and the like.

The term “aralkyl”, alone or in combination, means an alkyl or cycloalkyl radical as defined above in which one hydrogen atom is replaced by an aryl radical as defined above, such as benzyl, 2-phenylethyl, and the like.

The term “heterocyclic” means ring structures containing at least one heteroatom within the ring. As used herein, “heteroatom” refer to atoms that are neither carbon nor hydrogen bound to a carbon. Examples of heterocyclics include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups.

The term “saturated, partially saturated or unsaturated cyclic” means fused ring structures in which 2 carbons of the ring are also part of the fifteen-membered macrocyclic ligand. The ring structure can contain 3 to 20 carbon atoms, preferably 5 to 10 carbon atoms, and can also contain one or more other kinds of atoms in addition to carbon. The most common of the other kinds of atoms include nitrogen, oxygen and sulfur. The ring structure can also contain more than one ring.

The term “saturated, partially saturated or unsaturated ring structure” means a ring structure in which one carbon of the ring is also part of the fifteen-membered macrocyclic ligand.

The ring structure can contain 3 to 20, preferably 5 to 10, carbon atoms and can also contain nitrogen, oxygen and/or sulfur atoms.

The term “nitrogen containing heterocycle” means ring structures in which 2 carbons and a nitrogen of the ring are also part of the fifteen-membered macrocyclic ligand. The ring structure can contain 2 to 20, preferably 4 to 10, carbon atoms, can be substituted or unsubstituted, partially or fully unsaturated or saturated, and can also contain nitrogen, oxygen and/or sulfur atoms in the portion of the ring which is not also part of the fifteen-membered macrocyclic ligand.

The term “organic acid anion” refers to carboxylic acid anions having from about 1 to about 18 carbon atoms.

The term “halide” means chloride, floride, iodide, or bromide.

As used herein, “R” groups means all of the R groups attached to the carbon atoms of the macrocycle, i.e., R, R′, R₁, R′₁, R₂, R′₂, R₃, R′₃, R₄, R′₄, R₅, R′₅, R₆, R′₆, R₇, R′₇, R₈, R′₈, R₉, R′₉.

The mammal patient in the methods of the invention is a mammal suffering from hypotension associated with various shock states, including but not limited to septic shock, cardiogenic shock, burn-induced shock, anaphylactic shock and hypovolemic shock. The term “mammal suffering from hypotension” is contemplated to include cases in which hypotension is anticipated as well as cases in which hypotension is apparent. It is envisioned that a mammal patient to which the catalyst for the dismutation of superoxide will be administered, in the methods or compositions of the invention, will be a human. However, other mammal patients in veterinary (e.g., companion pets and large veterinary animals) and other conceivable contexts are also contemplated.

As used herein, the terms “treatment” or “treating” relate to any treatment of hypotension and include: (1) preventing hypotension from occurring in a subject; (2) inhibiting the fall of mean arterial pressure, i.e., arresting or limiting its development; or (3) ameliorating or relieving the symptoms of the disease.

All references cited herein are explicitly incorporated by reference.

### DETAILED DESCRIPTION

The present invention is directed to methods and compositions for the prevention and treatment of hypotension comprising administering compositions containing a catalyst for dismutation of superoxide. The composition can contain a catalyst for dismutation of superoxide alone or in combination with a catecholamine pressor agent. Preferred catalysts include superoxide dismutase enzyme (SOD) and small molecular weight organic ligand mimics of that enzyme (SOD mimetics or SODms).

A basis for the present invention is the finding that treatment with a catalyst for the dismutation of superoxide prevents the continued decrease in mean arterial pressure associated with hypotension such as that resulting from septic shock. While not being bound by any particular theory, applicants believe that superoxide (O₂⁻) reacts with catecholamines initiating a chain autooxidation reaction and deactivating them in vitro. Moreover, this deactivation appears to account for the hyporeactivity to exogenous catecholamines observed in cases of hypotension associated with septic shock and other shock conditions. This suggests that the deactivation of endogenous norepinephrine by O₂⁻ contributes significantly to this aspect of the vascular crisis. Thus, in one embodiment of the invention, the present methods and compositions use catalysts for the dismutation of superoxide to treat hypotension by removing O₂⁻, thereby protecting exogenous and endogenous catecholamines from autooxidation. As a result, both hyporeactivity and hypotension are reversed, and survival rate is improved.

It is preferred that non-proteinaceous catalysts for the dismutation of superoxide be used in the methods and compositions of the invention. The pentaaza-macrocyclic non-proteinaceous catalysts preferred for use in the invention have catalytic activities which are close to or equal that of the enzymatic catalysts. Unlike the enzymes, the non-proteinaceous catalysts do not degrade in solution when stored for long periods of time at ambient temperatures and are considerably less antigenic. In addition, these catalysts are usually much simpler to synthesize and produce than enzymes, which must be isolated from natural sources or produced using recombinant biotechnology.

Non-proteinaceous catalysts for the dismutation of superoxide preferred for use in the present invention preferably comprise an organic ligand chelated to a metal ion. Particularly preferred catalysts are pentaaza-macrocyclic ligand compounds, more specifically the manganese(II), manganese (II), iron(II) and iron(III) chelates of pentaazacyclopentadecanecompounds, which can be represented by the following formula:

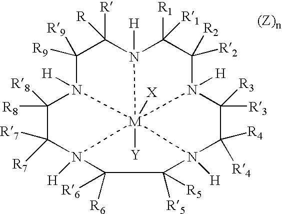

wherein M is a cation of a transition metal, preferably manganese or iron; wherein R, R′, R₁, R′₁, R₂, R′₂, R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R₁ or R′₁ and R₂ or R′₂, R₃ or R′₃ and R₄ or R′₄, R₅ or R′₅ and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; R or R′ and R₁ or R′₁, R₂ or R′₂ and R₃ or R′₂, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and one of R, R′, R₁, R′₁, R₂, R′₂, R₃, R′₃, R₄, R′₄, R₅, R′₅, R₆, R′₆, R₇, R′₇, R₈, R′₈, R₉ and R′₉ together with a different one of R, R′, R₁, R′₁, R₂, R′₂, R₃, R′₃, R₄, R′₄, R₅, R′₅, R₆, R′₆, R₇, R′₇, R₈, R′₈, R₉ and R′₉ which is attached to a different carbon atom in the macrocyclic ligand may be bound to form a strap represented by the formula:

—(CH2)ₓ-M-(CH2)_{w}-L-(CH2)_{z}—I—(CH2)_{y}—

wherein w, x, y and z independently are integers from 0 to 10 and M, L and I are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof.

Thus, the pentaaza-macrocyclic ligand compounds useful in the present invention can have any combinations of substituted or unsubstituted R groups, saturated, partially saturated or unsaturated cyclics, ring structures, nitrogen containing heterocycles, or straps as defined above.

X, Y and Z represent suitable ligands or charge-neutralizing anions which are derived from any monodentate or polydentate coordinating ligand or ligand system or the corresponding anion thereof (for example benzoic acid or benzoate anion, phenol or phenoxide anion, alcohol or alkoxide anion). X, Y and Z are independently selected from the group consisting of halide, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins. The preferred ligands from which X, Y and Z are selected include halide, organic acid, nitrate and bicarbonate anions.

The “R” groups attached to the carbon atoms of the macrocycle can be in the axial or equatorial position relative to the macrocycle. When the “R” group is other than hydrogen or when two adjacent “R” groups, i.e., on adjacent carbon atoms, together with the carbon atoms to which they are attached form a saturated, partially saturated or unsaturated cyclic or a nitrogen containing heterocycle, or when two R groups on the same carbon atom together with the carbon atom to which they are attached form a saturated, partially saturated or unsaturated ring structure, it is preferred that at least some of the “R” groups are in the equatorial position for reasons of improved activity and stability. This is particularly true when the complex contains more than one “R” group which is not hydrogen.

A wide variety of pentaaza-macrocyclic ligand compounds with superoxide dismutating activity may be readily synthesized. Generally, the transition metal center of the catalyst is thought to be the active site of catalysis, wherein the manganese or iron ion cycles between the (II) and (III) states. Thus, as long as the redox potential of the ion is in a range in which superoxide anion can reduce the oxidized metal and protonated superoxide can oxidize the reduced metal, and steric hindrance of the approach of the superoxide anion is minimal, the catalyst will function with a k_{cat} of about 10⁻⁶ to 10⁻⁸.

The pentaaza-macrocyclic ligand compound catalysts described have been further described in U.S. Pat. No. 5,637,578, PCT application WO98/58636, and copending application U.S. Ser. No. 09/398,120, all of which are hereby incorporated by reference. These pentaaza-macrocyclic ligand catalysts may be produced by the methods disclosed in U.S. Pat. No. 5,610,293. However, it is preferred that the pentaaza-macrocyclic ligand compound catalysts used in the present invention be synthesized by the template method described in copending applications U.S.S. No. 60/136,298 and U.S. Ser. No. 09/398,120, incorporated herein by reference.

Also suitable for use in the present invention, but less preferred than the pentaaza-macrocyclic ligand compounds, are the salen complexes of manganese and iron disclosed in U.S. Pat. No. 5,696,109, here incorporated by reference. The term salen complex means a ligand complex with the general formula:

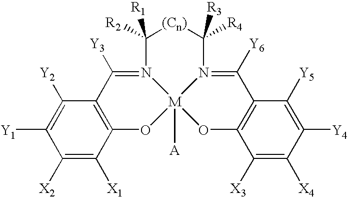

wherein M is a transition metal ion, preferably manganese or iron; A is an anion, typically Cl; and n is either 0, 1, or 2. X₁, X₂, X₃ and X₄ are independently selected from the group consisting of hydrogen, silyls, arlyls, aryls, arylalkyls, primary alkyls, secondary alkyls, tertiary alkyls, alkoxys, aryloxys, aminos, quaternary amines, heteroatoms, and hydrogen; typically X₁ and X₃ are from the same functional group, usually hydrogen, quaternary amine, or tertiary butyl, and X₂ and X₄ are typically hydrogen. Y₁, Y₂, Y₃, Y₄, Y₅ and Y₆ are independently selected from the group consisting of hydrogen, halides, alkyls, aryls, arylalkyls, silyl groups, aminos, alkyls or aryls bearing heteroatoms; aryloxys, alkoxys, and halide; preferably, Y₁ and Y₄ are alkoxy, halide, or amino groups. Typically, Y₁ and Y₄ are the same. R₁, R₂, R₃ and R₄ are independently selected from the group consisting of H, CH₃, C₂H₅, C₆H₅, O-benzyl, primary alkyls, fatty acid esters, substituted alkoxyaryls, heteroatom-bearing aromatic groups, arylalkyls, secondary alkyls, and tertiary alkyls. Methods of synthesizing these salen complexes are also disclosed in U.S. Pat. No. 5,696,109.

Iron or manganese porphyrins, such as, for example, Mn^{III} tetrakis(4-N-methylpyridyl)porphyrin, Mn^{III}tetrakis-o-(4-N-methylisonicotinamidophenyl)porphyrin, Mn^{III}tetrakis(4-N—N—N-trimethylanilinium)porphynin, Mn^{III}tetrakis(1-methyl-4-pyridyl)porphyrin, Mn^{III} tetrakis(4-benzoic acid)porphyrin, Mn^{II}octabromo-meso-tetrakis(N-methylpyridinium-4-yl)porphyrin, 5, 10, 15, 20-tetrakis (2,4,6-trimethyl-3,5-disulfonatophenyl)-porphyrinato iron (III) (FeTMPS), Fe^{III}tetrakis(4-N-methylpyridyl)porphyrin, and Fe^{III}tetrakis-o-(4-N-methylisonicotinamidophenyl)porphyrin and preferably, substituted iron porphyin 5,10,15,20-tetrakis(2,4,6-trimethyl-3,5-disulfonatophenyl)-porphyrinato iron (III) (FeTMPS) may also be used in the methods and compositions of the present invention. See U.S. Pat. No. 6,103,714. The catalytic activities and methods of purifying or synthesizing these non-proteinaceous catalysts are well known in the organic chemistry arts.

In addition to non-proteinaceous catalysts for the dismutation of superoxide, superoxide dismutatse enzymes (SODs) isolated from various sources, or recombinantly produced, may be used in the methods and compositions of the present invention. The best known of these enzymes is CuZn SOD, which is a dimer with a molecular weight of 33,000 containing two copper and two zinc atoms. CuZn SOD is found in the cytosol and in the intermembrane space of the mitochondria. Mn SOD is a tetramer with a molecular weight of 85,000 containing 4 Mn atoms, and is mainly located in the mitochondrial matrix. These enzymes are well known in the biochemical arts, and methods for their isolation and preparation are also well known. See U.S. Pat. No. 5,788,961, incorporated herein by reference. In addition, CuZn SOD is commercially available under the trade name ORGOTEIN (Peroxinorm). As the method of action of the invention is presumed to take place in the walls of the blood vessels of the mammal, the difference in diffusion rates between these enzyme catalysts and the non-proteinaceous catalysts would not be expected to affect the catecholamine preservation effect seen with the intravascular administration of catalysts for the dismutation of superoxide. Thus, these enzyme catalysts would be expected to be effective in the methods and compositions of the invention. However, enzyme catalysts are not preferred, as they can cause allergic reactions in some individuals, are fairly rapidly degraded in the bloodstream, and are much more difficult to produce than their small organic ligand non-proteinaceous counterparts.

Activity of the compounds or complexes of the present invention for catalyzing the dismutation of superoxide can be demonstrated using the stopped-flow kinetic analysis technique as described in Riley, D. P. et al., Anal. Biochein., 196: 344-349 (1991) which is incorporated herein by reference. Stopped-flow kinetic analysis is an accurate and direct method for quantitatively monitoring the decay rates of superoxide in water. The stopped-flow kinetic analysis is suitable for screening compounds for SOD activity and activity of the compounds or complexes of the present invention, as shown by stopped-flow analysis, correlate to treating the above disease states and disorders.

Contemplated equivalents of the general formulas set forth above for the compounds and derivatives as well as the intermediates are compounds otherwise corresponding thereto and having the same general properties such as tautomers of the compounds and such as wherein one or more of the various R groups are simple variations of the substituents as defined therein, e.g., wherein R is a higher alkyl group than that indicated, or where the tosyl groups are other nitrogen or oxygen protecting groups or wherein the O-tosyl is a halide. Anions having a charge other than 1, e.g., carbonate, phosphate, and hydrogen phosphate, can be used instead of anions having a charge of 1, so long as they do not adversely affect the overall activity of the complex. However, using anions having a charge other than 1 will result in a slight modification of the general formula for the complex set forth above. In addition, where a substituent is designated as, or can be, a hydrogen, the exact chemical nature of a substituent which is other than hydrogen at that position, e.g., a hydrocarbyl radical or a halogen, hydroxy, amino and the like functional group, is not critical so long as it does not adversely affect the overall activity and/or synthesis procedure. Further, it is contemplated that manganese(III) complexes will be equivalent to the subject manganese(II) complexes.

In a preferred embodiment, catalysts for the dismutation of superoxide are coupled with catecholamine pressor agents to be used in the methods and compositions of the invention. Preferably, the catecholamine pressor agent is dopamine, norepinephrine, epinephrine and alpha agonist phenyleprine, more preferably, dopamine and norepinephrine. Without being bound to any particular theory, applicants propose that the administration of a composition comprising a catalyst for dismutation of superoxide and a catecholamine pressor agent to a mammal suffering from hypotension will prevent the degradation of the catecholamines, thus allowing the catecholamine pressor agent to improve vascular tone and increase the mean arterial blood pressure of the mammal.

Pharmaceutical Compositions

For use in treatment or prophylaxis of mammals, the compounds of the invention can be formulated as pharmaceutical or veterinary compositions. Depending on the subject to be treated, the mode of administration, and the type of treatment desired (e.g., inhibition, prevention, prophylaxis, therapy), the compounds are formulated in ways consonant with these parameters. The compositions of the present invention comprise a therapeutically or prophylactically effective dosage of a catalyst for the dismutation of superoxide. The catalyst for the dismutation of superoxide is preferably a superoxide dismutase enzyme such as CuZn SOD, or a small molecular weight organic ligand mimics of that enzyme (SODm). In a preferred embodiment, the catalyst is a non-proteinaneous catalyst comprising an organic ligand and a transitional metal cation, more preferably manganese(II), manganese (III), iron (II), and iron(III) chelates of pentaazacyclopentadecane compounds. Also suitable for use in the present invention are the salen complexes of manganese and iron disclosed in U.S. Pat. No. 5,696,109, and iron or manganese porphyrins as discussed above.

In another embodiment of the invention, pharmaceutical or veterinary compositions are provided which comprise catalysts for the dismutation of superoxide and catecholamine pressor agents.

When administered to a mammal suffering from hypotension, these pharmaceutical compositions prevent the degradation of the catecholamines, allowing the catecholamine pressor agent to improve vascular tone and increase the mean arterial blood pressure of the mammal.

The compositions of the present invention may be incorporated in conventional pharmaceutical formulations (e.g. injectable solutions) for use in treating humans or animals in need thereof. Pharmaceutical compositions can be administered by subcutaneous, intravenous, or intramuscular injection, or as large volume parenteral solutions and the like. The term parenteral as used herein includes subcutaneous injections, intravenous, intramuscular, intrastemal injection, or infusion techniques.

For example, a parenteral therapeutic composition may comprise a sterile isotonic saline solution containing between 0.1 percent and 90 percent weight to volume of the catalysts for the dismutation of superoxide. A preferred solution contains from about 5 percent to about 20 percent, more preferably from about 5 percent to about 17 percent, more preferably from about 8 to about 14 percent, and most preferably about 10 percent catalysts for dismutation of superoxide in solution (% weight per volume).

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols which are solid at room temperature but liquid at the rectal temperature and will therefore melt in the rectum and release the drug.

Solid dosage forms for oral administration may include capsules, tablets, pills, powders, granules and gels. In such solid dosage forms, the active compound may be admixed with at least one inert diluent such as sucrose lactose or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

For administration to animal or human subjects, a typical dose of the composition comprising a catalyst for the dismutation of superoxide and a catecholamine pressor agent can be from about 0.001 to about 10 milligrams of active composition per kilogram of patient body weight. Preferably, the dosage will range between 0.001 to 5 mg/kg patient body weight, more preferably 0.05 to 5 mg/kg body weight, and most preferably 0.05 to 1 mg/kg body weight. Thus, a typical dose for a human patient might be from a milligram to over 75 milligrams; the dosages for a companion pet such as a dog or cat will be less than 7 millgrams; and the dosages for large veterinary animals will be more than 500 milligrams. Total daily dose may be administered to a mammal in single or divided doses may be in amounts, for example, from about 1 to about 2 mg/kg body weight daily and more usually about 0.05 to 1 mg/kg. Dosage unit compositions may contain such amounts of submultiples thereof to make up the total dose. However, one skilled in the art will recognize that the total dosage will vary on the particular composition comprising a catalyst for the dismutation of superoxide and a catecholamine pressor agent being administered.

The amount of active ingredient that may be combined with the carrier materials to produce a single dosage form will vary depending upon the host treated and the particular mode of administration. It will be appreciated that the unit content of active ingredients contained in an individual dose of each dosage form need not in itself constitute an effective amount, as the necessary effective amount could be reached by administration of a number of individual doses. The selection of dosage depends upon the dosage form utilized, the condition being treated, and the particular purpose to be achieved according to the determination of those skilled in the art.

The dosage regimen for treating a disease condition with the compounds and/or compositions of this invention is selected in accordance with a variety of factors, including the type, age, weight, sex, diet and medical condition of the patient, the route of administration, pharmacological considerations such as the activity, efficacy, pharmacokinetic and toxicology profiles of the particular compound employed, whether a drug delivery system is utilized and whether the compound is administered as part of a drug combination. Thus, the dosage regimen actually employed may vary widely and therefore may deviate from the preferred dosage regimen set forth above.

The following examples are offered in order to illustrate but not to limit the present invention.

### EXAMPLES

### Example 1

### Materials and Methods

Anaesthetized Rat Model. Male Sprague Dawley rats (250-300 g) were anaesthetized with inactin (100 mg/Kg intraperitoneally). The trachea was cannulated to facilitate respiration and body temperature was maintained at 37° C. by means of a heating pad. The left femoral vein was cannulated for administration of drugs. The left femoral artery was cannulated and connected to a pressure transducer to allow for the monitoring of blood pressure. Lipopolysaccharide from E. coli (LPS; 4 mg/Kg, serotype 0111:B4) was administered as a bolus intravenous injection at a volume of 0.3 ml. Control animals received saline at the same volume and by the same route. In experiments involving blood samples, such blood samples were withdrawn from the arterial cannula.

Catecholamine measurements. Catecholamines in test tube samples or plasma samples were identified and quantified by high pressure liquid chromatography with electrochemical detection (HPLC-EC). The system consists of a Varian model 2510 solvent delivery system and a model 9090 autosampler (Varian, Walnut Creek, Calif.) coupled to a C18 column and an ESA Coulochem II detector. Separations were performed isocratically using a filtered and degassed mobile phase consisting of 10% methanol, 0.1 M sodium phosphate, 0.2 mM sodium octyl sulfate and 0.1 mM EDTA, adjusted to pH 2.8 with phosphoric acid. The HPLC system is coupled to a PS-90 computer with which chromatograms were recorded and analyzed with Varian Star workstation software.

Adrenochrome measurements. The detection and quantification of the sum of the noradrenochrome and adrenochrome was carried out using an HPLC method utilising a Vydac C18 Pharmaceutical 4.6×250 mm column and with a 5% acetonitrile+95% SDS (10 mM) mobile phase (5 min elution), then 40% acetonitrile with 60% SDS plus 0.1% TFA (5 min elution) mobile phase, all eluted at 1 mil/min. Detection of the adrenochromes utilizes the visible fluorescence of their adrenolutin product formed via treatment with NaOH (1 M, 1 ml/min) as post column derivatization. The resultant adrenolutins are detected via the emission at 518 nm following excitation at 406 nm with linear detection response to ppb levels. Because the adrenochromes are unstable in plasma at 37° C. (reacting in a 1^{st}-order fashion with a t_{1/2} of 21 min with the nucleophilic components of the plasma proteins), it is important to slow this process by cooling the blood samples to 2-4° C. and maintain that low temperature for all subsequent handling. The blood samples are processed in the following manner: 100 ml of cell free plasma (obtained via centrifugation of the blood at 4° C. to separate the cells) is added to 300 ml acetonitrile and centrifuged at 4° C. to precipitate proteins. The supernatant is then injected directly (100 ml).

Statistics. Statistical differences between treatments were determined by one-way analysis of variance, followed by Student-Newman-Keuls test. Statistical differences were accepted when P<0.05.

### Example 2

### In Vivo Evaluation

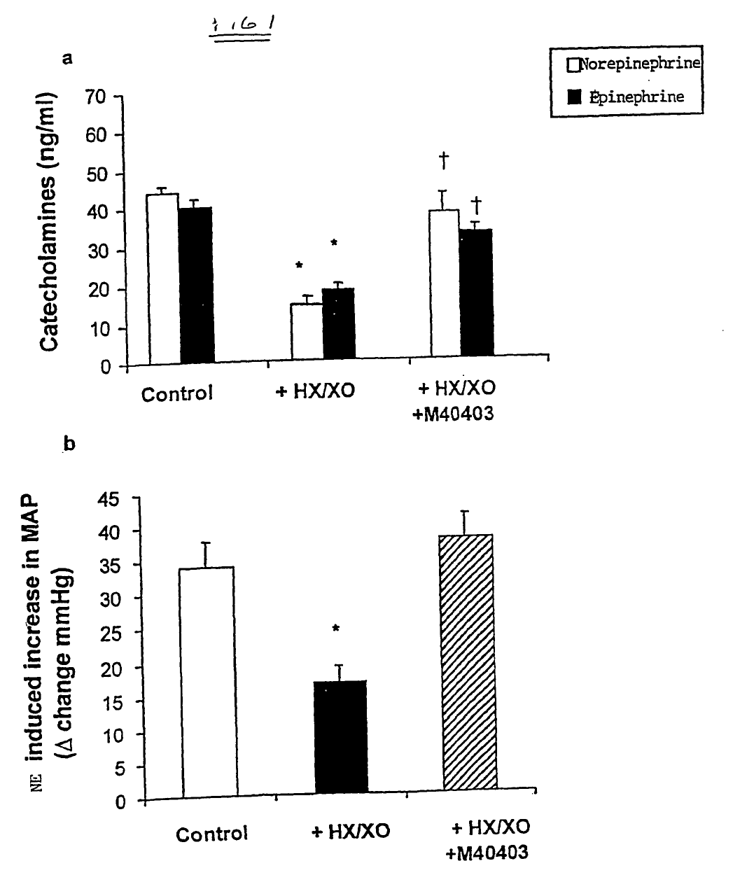

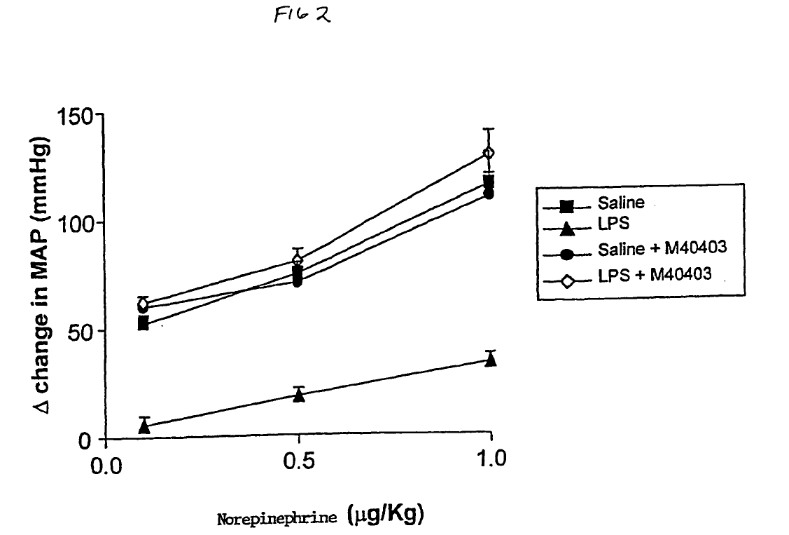

Hypoxanthine (HX;2 mM)/xanthine oxidase (XO; 1 U/ml) results in the generation of O₂⁻ in the ratio of 2 molecules of O₂⁻ to every one molecule of HX used. Exposing synthetic catecholamines (norepinephrine and epinephrine) to this superoxide generating system resulted in significant decreases in the chemical detection of the catecholamines by HPLC (FIG. 1a; n=6). These decreases were prevented by the presence of the SOD mimetic M40403 (FIG. 1a; n=6). This data suggests that O₂⁻ is reacting with the catecholamines and converting them to non-catecholamine products which have been identified by HPLC as adrenochromes.

Male Sprague Dawley rats (250-300 g, 6 rats per group) were anaesthetized and prepared according to the methods of Example 1. 0.5 mg/Kg of norepinephrine was administered as an intravenous (i.v) bolus injection through the left femoral vein. Animals in a control group received saline at the same volume and by the same route. The left femoral artery was cannulated and connected to a pressure transducer to allow for the monitoring of blood pressure during the experiment. The change in the blood pressure of the animals was compared to the blood pressure of animals in the control group. The results of the tests can be found in FIG. 1. Norepinephrine raised the MAP of the rats by 34±3.7 mmHg (FIG. 1b; n=6). After incubation with HX/XO (which has no effect on MAP by itself), the ability of norepinephrine to increase MAP was significantly attenuated (from 34±3.7 mmhg to 17±2.5 mmHg; FIG. 1b; n=6). When SOD mimetic M40403 was included with HX/XO in the incubation mixture, the vasopressor actions of were protected as shown by its ability to restore MAP back to near control values (38±3.6 mmHg) (FIG. 1b; n=6). These data clearly demonstrate that O₂⁻ can deactivate norepinephrine in vitro and, as a consequence, abolish its biological activity as evidenced by the loss of its vasopressor effects in vivo.

### Example 3

### Effect of Norepinephrine and LRP on MAP

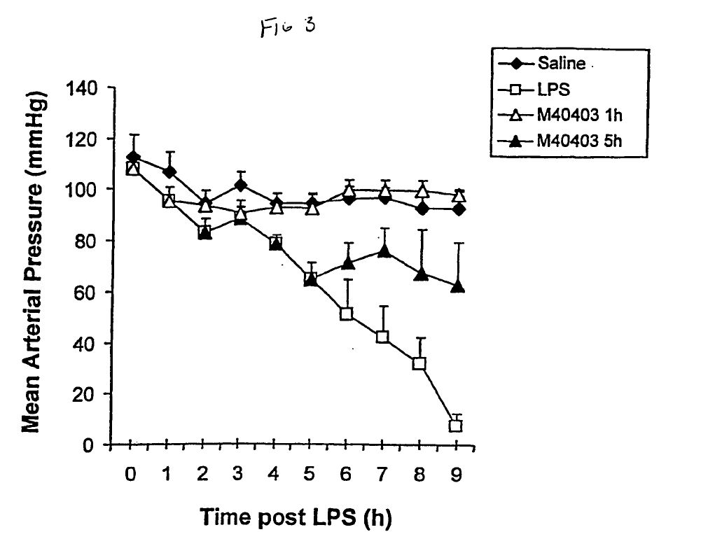

Injection of E. coli lipopolysaccharide (LPS) to rats leads to the development of hyporeactivity to exogeneously administered norepinephrine which typically occurs in the first two hours. Male Sprague Dawley rats (250-300 g, number in group) were anaesthetized and prepared according to the methods of Example 1. Increasing dosages of norepinephrine (0.1, 0.5 and 1 mg/Kg) were given as intravenous bolus injections. Animals in a control group received saline at the same volume and by the same route. The left femoral artery was cannulated and connected to a pressure transducer to allow for the monitoring of blood pressure during the experiment. The change in the blood pressure of the animals was compared to the blood pressure of animals in the control group. As can be seen in FIG. 2, the mean arterial pressure (MAP) of anaesthetized rats increased in a dose dependent manner. Two hours after the injection of LPS (4 mg/Kg), the pressor responses to norepinephrine (0.1, 0.5 and 1 mg/Kg) were greatly reduced, indicative of the development of hyporeactivity (FIG. 2; n=6): these responses to norepinephrine were restored by SOD mimetic M40403 (0.25 mg/Kg, given as a 15 min i.v infusion 1 hour after LPS, FIG. 2). Pressor responses to norepinephrine in rats not treated with LPS were unaffected by the SOD mimetic M40403 (FIG. 2; n=6). These data strongly support our hypothesis that the hyporeactivity that develops in sepsis to exogenously administered norepinephrine is caused by the deactivation of this catecholamine by O₂⁻ produced in vivo.

### Example 4

### Effect of Administration of LPS and SOD Mimetic M40403

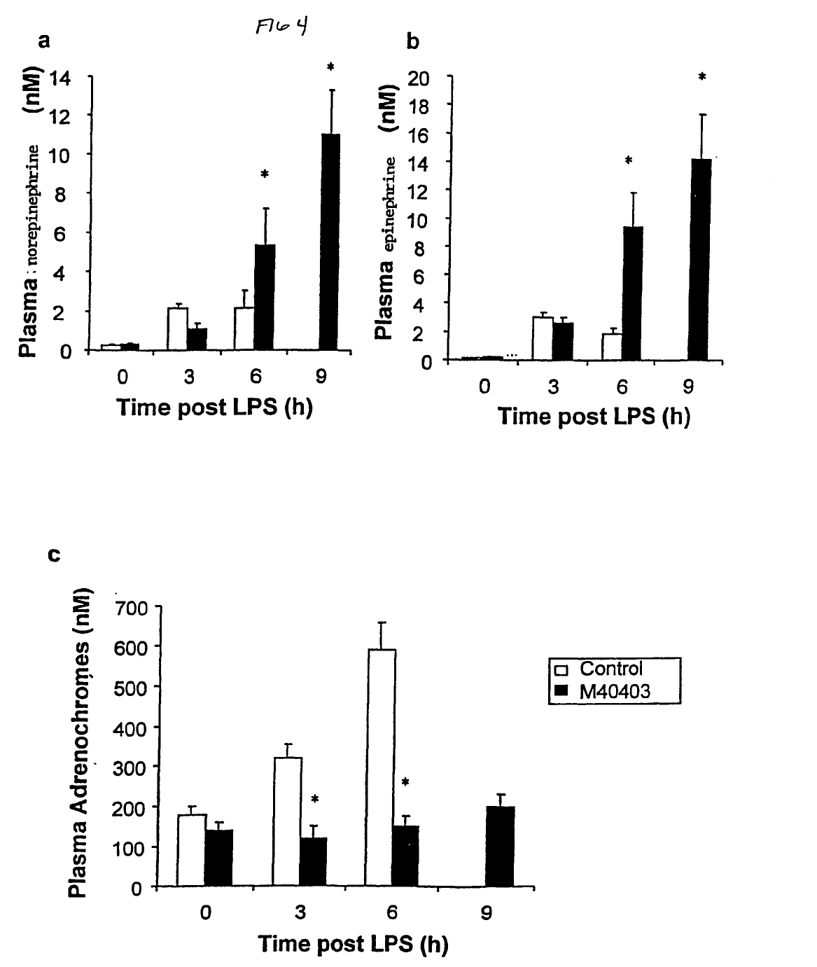

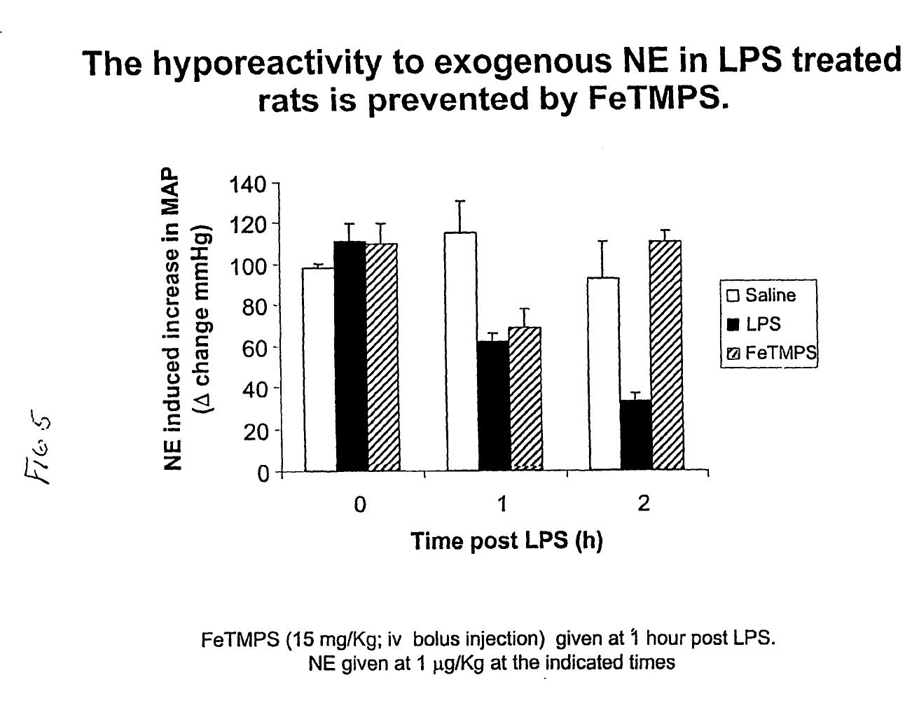

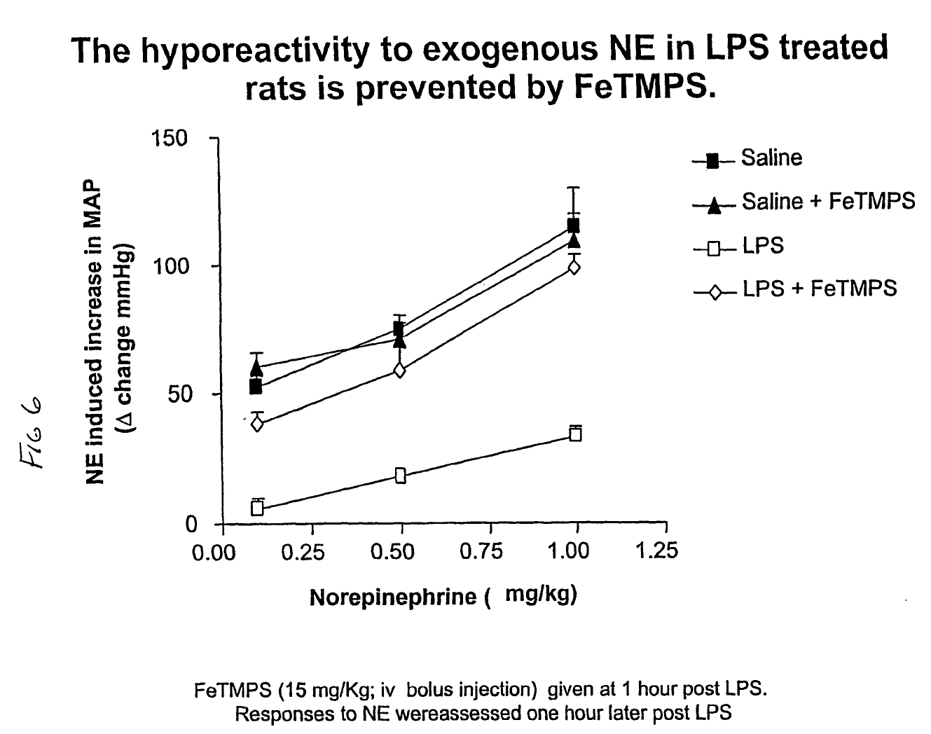

Intravenous injection of LPS (4 mg/Kg) in rats led to a profound fall in blood pressure associated with a high mortality rate (99±5% mortality at 9 hours, n=10, FIG. 3). Furthermore, the plasma levels of norepinephrine as well as the adrenochromes increased after LPS treatment (FIGS. 4a,b,c; n=10). Levels of the catecholamines and of the adrenochromes could not be evaluated at the 9 h timepoint (survival rate at this point was 1%, n=10).

When M40403 (0.25 mg/Kg/h) was administered as an i.v. infusion 1 hour post LPS for the duration of the experimental protocol, the development of hypotension was prevented and mortality rate greatly reduced (99±2% survival by 9 h, n=10). (FIG. 3; n=10). Inhibition of hypotension by M40403 was associated with increased levels of catecholamines (FIGS. 4a and b) and concommittant decrease (FIG. 4c; n=10) in plasma levels of the adrenochromes, the reaction products of O₂⁻ and these catecholamines. In addition, when the administration of M40403 was postponed until 5 hours post LPS in this model the severe hypotensive phase of this condition was reversed (FIG. 3; n=10).

### Example 5

### Effect of Administration of FeTMPS on Exogenous NE

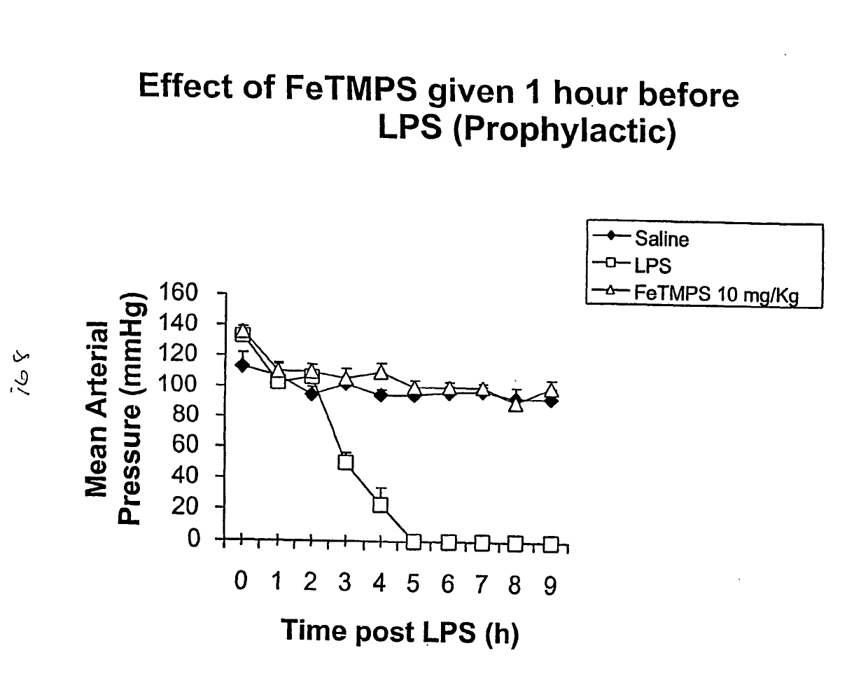

Injection of E. coli lipopolysaccharide (LPS) to rats leads to the development of hyporeactivity to exogeneously administered norepinephrine which typically occurs in the first two hours. Male Sprague Dawley rats (250-300 g, number in group) were anaesthetised and prepared according to the methods of Example 1. Increasing dosages of norepinephrine (0.1, 0.5 and 1 mg/Kg) were given as intravenous bolus injections. Animals in a control group received saline at the same volume and by the same route. The left femoral artery was cannulated and connected to a pressure transducer to allow for the monitoring of blood pressure during the experiment. The change in the blood pressure of the animals was compared to the blood pressure of animals in the control group. As can be seen in FIG. 6, the change in mean arterial pressure induced by NE increased in a dose dependent manner. Two hours after the injection of LPS (4 mg/Kg), the pressor responses to norepinephrine (0.1, 0.5 and 1 mg/Kg) were greatly reduced, indicative of the development of hyporeactivity.

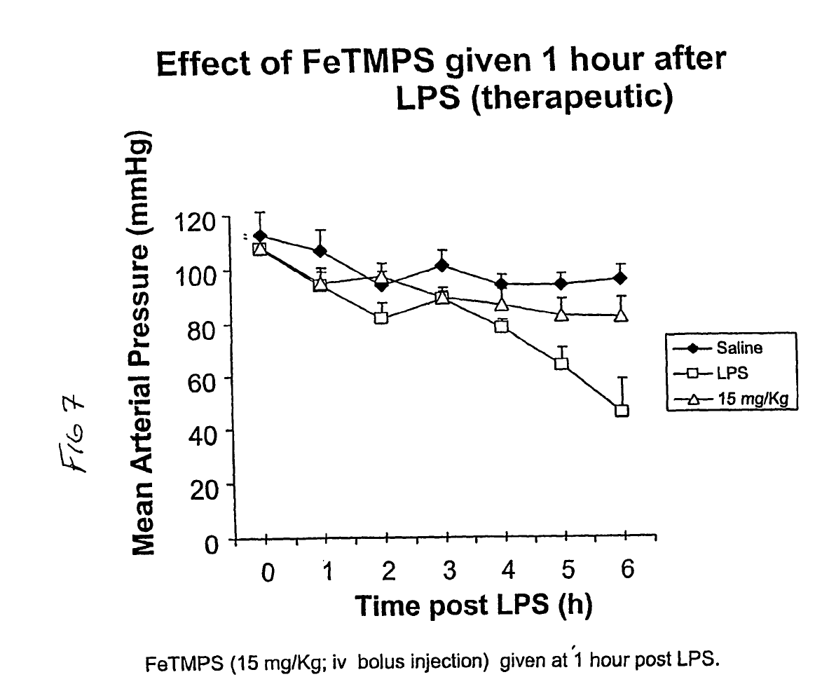

When 15 mg/Kg of 5,10,15,20-tetrakis (2,4,6-trimethyl-3,5-disulfonatophenyl)-porphyrinato iron (III) (FeTMPS) was administered (i.v bolus injection 1 hour after LPS), the development of hypotension was prevented. See FIGS. 5-8.

These findings provide strong evidence for a pivotal role for the deactivation of catecholamines by 2- and suggest that the hyporeactivity to exogenous norepinephrine observed may be explained by the fact that patients basically receive a vasoconstrictor that is deactivated through in vivo generation of O₂⁻. Furthermore, these results also indicate that the deactivation of endogenous vasoconstrictor catecholamines may contribute significantly to severe hypotension.

Other features, objects and advantages of the present invention will be apparent to those skilled in the art. The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the present invention.

We claim:

1. A method for inhibiting a fall in mean arterial pressure in a mammal suffering from hypotension, the method comprising administering to the mammal a mean arterial pressure sustaining amount of a composition comprising a catalyst for the dismutation of superoxide.

2. The method of claim 1 wherein inhibition of the fall in mean arterial pressure is achieved by limiting autooxidation of catecholamines.

3. The method of claim 2 wherein the catalyst is a non-proteinaceous catalyst comprising an organic ligand chelated to a metal ion selected from the group of manganese(II), manganese(III), iron(II) and iron(III).

4. The method of claim 3, wherein the catalyst is a pentaaza-macrocyclic ligand complex.

5. The method of claim 4 wherein the pentaaza-macrocyclic ligand complex is selected from the group consisting of manganese and iron chelates of pentaazacyclopentadecane compounds, which are represented by the following formula: 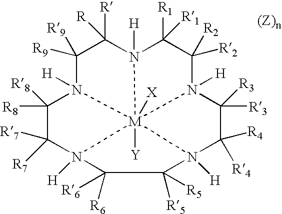
wherein M is a cation of a transition metal, preferably manganese or iron; wherein R, R′, R₁, R′₁, R₂, R′₂, R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R₁ or R′₁ and R₂ or R′₂, R₃ or R′₃ and R₄ or R′₄, R₅ or R′₅ and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and one of R, R′, R₁, R′₁, R₂, R′₂, R₃, R′₃, R₄, R′₄, R₅, R′₅, R₆, R′₆, R₇, R′₇, R₈, R′₈, R₉, and R′₉ together with a different one of R, R′, R₁, R′₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula
—(CH₂)ₓ-M-(CH₂)_{w}-L-(CH₂)_{z}—I—(CH₂)_{y}—
wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and combinations thereof;
and wherein X, Y and Z are independently selected from the group consisting of halide, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins.

6. The method of claim 3, wherein the catalyst is a porphyrin ligand complex or a substituted porphyrin ligand complex.

7. The method of claim 6 wherein the porphyrin ligand complex is selected from the group consisting of manganese (II) porphyrin complexes, manganese(III) porphyrin complexes, iron (II) porphyrin complexes, and iron(III) porphyrin complexes.

8. The method of claim 7 wherein the porphyrin ligand complex is a 5,10,15,20-tetrakis (2,4,6-trimethyl-3,5-disulfonatophenyl)-porphyrinato iron (III) (FeTMPS).

9. The method as in either claim 1 or 3, wherein the hypotension results from septic shock.

10. The method as in either claim 1 or 3, wherein the hypotension results from cardiogenic shock.

11. The method as in either claim 1 or 3, wherein the hypotension results from burn-induced shock.

12. The method as in either claim 1 or 3, wherein the hypotension results from hypovolemic shock.

13. The method as in either claim 1 or 3, wherein the hypotension results from anaphylactic shock.

14. The method as in either claim 1 or 3, wherein the mammal is a human.

15. The method as in either claim 1 or 3, wherein the mammal is a companion pet.

16. The method as in either claim 1 or 3, wherein the mammal is a large veterinary animal.

17. The method as in either claim 1 or 3, wherein the catalyst is administered by intraarterial, intravenous, intramuscular or subcutaneous injection.

18. A method for increasing mean arterial pressure in a mammal suffering from hypotension, the method comprising administering to the mammal a mean arterial pressure increasing amount of a composition comprising a catecholamine pressor agent and a catalyst for the dismutation of superoxide.

19. The method of claim 18 wherein inhibition of the fall in mean arterial pressure is achieved by limiting autooxidation of catecholamines.

20. The method of claim 19, wherein the catalyst is a non-proteinaceous catalyst, and the catalyst comprises an organic ligand chelated to a metal ion selected from the group of manganese(II), manganese(III), iron(II) and iron(III).

21. The method of claim 20, wherein the catalyst is a pentaaza-macrocyclic ligand complex.

22. The method of claim 21 wherein the pentaaza-macrocyclic ligand complex is selected from the group consisting of manganese and iron chelates of pentaazacyclopentadecane compounds, which are represented by the following formula: 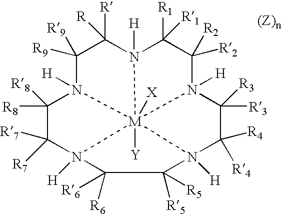
wherein M is a cation of a transition metal, preferably manganese or iron; wherein R, R′, R₁, R′₁, R₂, R′₂, R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R₁ or R′₁, and R₂ or R′₂, R₃ or R′₃ and R₄ or R′₄, R₅ or R′₅ and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and one of R, R′, R₁, R′₁, R₂, R′₂, R₃, R′₃, R₄, R′₄, R₅, R′₅, R₆, R′₆, R₇, R′₇, R₈, R′₈, R₉, and R′₉ together with a different one of R, R′, R₁, R′₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula
—(CH₂)ₓ-M-(CH₂)_{w}-L-(CH₂)_{z}—I—(CH₂)_{y}—
wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and combinations thereof;
and wherein X, Y and Z are independently selected from the group consisting of halide, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbarnate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins.

23. The method of claim 20, wherein the catalyst is a porphyrin complex or a substituted porphyrin complex.

24. The method of claim 23 wherein the porphyrin complex is selected from the group consisting of manganese (II) porphyrin complexes, manganese(III) porphyrin complexes, iron (I) porphyrin complexes, and iron(II) porphyrin complexes.

25. The method of claim 24 wherein the porphyrin ligand complex is a 5,10,15, 20-tetrakis(2,4,6-trimethyl-3,5-disulfonatophenyl)-porphyrinato iron (III) (FeTMPS).

26. The method as in either claim 18 or 20, wherein the catecholamine pressor agent is selected from the group consisting of dopamine, norepinephrine and epinephrine.

27. The method as in either claim 18 or 20, wherein the hypotension results from septic shock.

28. The method as in either claim 18 or 20, wherein the hypotension results from cardiogenic shock.

29. The method as in either claim 18 or 20, wherein the hypotension results from burn-induced shock.

30. The method as in either claim 18 or 20, wherein the hypotension results from anaphylactic shock.

31. The method as in either claim 18 or 20, wherein the mammal is a human.

32. The method as in either claim 18 or 20, wherein the mammal is a companion pet.

33. The method as in either claim 18 or 20, wherein the mammal is a large veterinary animal.

34. The method as in either claim 18 or 20, wherein the catalyst is administered by intraarterial, intravenous, intramuscular or subcutaneous injection.

35. The method as in either claim 18 or 20, wherein the catalyst is administered before the administration of the catecholamine.

36. The method as in either claim 18 or 20, wherein the catalyst is administered contemporaneously with the catecholamine.

37. A pharmaceutical composition comprising a catalyst for the dismutation of superoxide and a catecholamine pressor agent in a pharmaceutically acceptable carrier.

38. The composition of claim 37, wherein the catalyst is a non-proteinaceous catalyst, and the catalyst comprises an organic ligand chelated to a metal ion selected from the group of manganese(II), manganese(III), iron(II) and iron(III).

39. The composition of claim 38, wherein the catalyst is a pentaaza-macrocyclic ligand complex.

40. The composition of claim 39 wherein the pentaaza-macrocyclic ligand complex is selected from the group consisting of manganese and iron chelates of pentaazacyclopentadecane compounds, which are represented by the following formula: 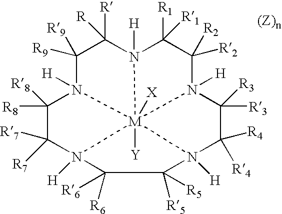
wherein M is a cation of a transition metal, preferably manganese or iron; wherein R, R′, R₁, R′₁, R₂, R′₂, R₃, R′₃, R₄, R′₄, R₅, R′₅, R₆, R′₆, R₇, R′₇, R₈, R′₈, P, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R₁ or R′₁, and R₂ or R′₂, R₃ or R′₃ and R₄ or R′₄, R₅ or R′₅ and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and one of R, R′, R₁, R′₁, R₂, R′₂, R₃, R′₃, R₄, R′₄, R₅, R′₅, R₆, R′₆, R₇, R′₇, R₈, R′₈, R₉, and R′₉ together with a different one of R, R′, R₁, R′₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula
—(CH₂)ₓ-M-(CH₂)_{w}-L-(CH₂)_{z}—I—(CH₂)_{y}—
wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and combinations thereof;
and wherein X, Y and Z are independently selected from the group consisting of halide, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins.

41. The composition of claim 38, wherein the catalyst is a porphyrin ligand complex or a substituted porphyrin ligand complex.

42. The composition of claim 41, wherein the porphyrin ligand complex is selected from the group consisting of manganese (II) porphyrin complexes, manganese(II) porphyrin complexes, iron (II) porphyrin complexes, and iron(III) porphyrin complexes.

43. The composition of claim 42 wherein the porphyrin ligand complex is a 5,10,15, 20-tetrakis(2,4,6-trimethyl-3,5-disulfonatophenyl)-porphyrinato iron (III) (FeTMPS).

44. The composition of claim 38, wherein the catecholamine pressor agent is selected from the group consisting of dopamine, norepinephrine, and epinephrine.

45. A method for treatment or prophylaxis of cardiogenic shock by inhibiting hypotension in a mammal, said method comprising administering to the mammal a mean arterial pressure sustaining amount of a catalyst for the dismutation of superoxide.

46. The method of claim 45 wherein the catalyst is a non-proteinaceous catalyst, and the non proteinaceous catalyst comprises an organic ligand chelated to a metal ion selected from the group of manganese(II), manganese(III), iron(II) and iron(III).

47. The method of claim 46, wherein the catalyst is a pentaaza-macrocyclic ligand complex.

48. The method of claim 47, wherein the pentaaza-macrocyclic ligand complex is selected from the group consisting of manganese and iron chelates of pentaazacyclopentadecane compounds, which are represented by the following formula: 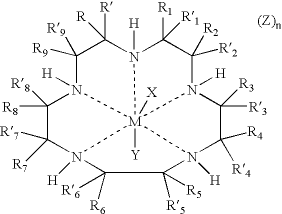
wherein M is a cation of a transition metal, preferably manganese or iron; wherein R, R′, R₁, R′₁, R₂, R′₂, R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R₁ or R′₁, and R₂ or R′₂, R₃ or R′₃ and R₄ or R′₄, R₅ or R′₅ and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and one of R, R′, R₁, R′₁, R₂, R′₂, R₃, R′₃, R₄, R′₄, R₅, R′₅, R₆, R′₆, R₇, R′₇, R₈, R′₈, R₉, and R′₉ together with a different one of R, R′, R₁, R′₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula
—(CH₂)ₓ-M-(CH₂)_{w}-L-(CH₂)_{z}—I—(CH₂)_{y}—
wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and combinations thereof;
and wherein X, Y and Z are independently selected from the group consisting of halide, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins.

49. The method of claim 46, wherein the catalyst is a porphyrin ligand complex or a substituted porphyrin ligand complex.

50. The method of claim 49, wherein the porphyrin ligand complex is selected from the group consisting of manganese (II) porphyrin complexes, manganese(III) porphyrin complexes, iron (II) porphyrin complexes, and iron(III) porphyrin complexes.

51. The method of claim 50 wherein the porphyrin ligand complex is a 5,10,15, 20-tetrakis(2,4,6-trimethyl-3,5-disulfonatophenyl)-porphyrinato iron (III) (FeTMPS).

52. A method for treatment or prophylaxis of burn-induced shock by inhibiting hypotension in a mammal, said method comprising administering to the mammal a mean arterial pressure sustaining amount of a composition comprising a catalyst for the dismutation of superoxide.

53. The method of claim 52 wherein the catalyst is a non-proteinaceous catalyst, and the non proteinaceous catalyst comprises an organic ligand chelated to a metal ion selected from the group of manganese(II), manganese(III), iron(II) and iron(III).

54. The method of claim 53, wherein the catalyst is a pentaaza-macrocyclic ligand complex.

55. The method of claim 54, wherein the pentaaza-macrocyclic ligand complex is selected from the group consisting of manganese and iron chelates of pentaazacyclopentadecane compounds, which are represented by the following formula: 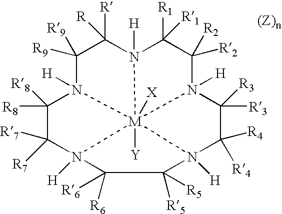
wherein M is a cation of a transition metal, preferably manganese or iron; wherein R, R′, R₁, R′₁, R₂, R′₂, R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R₁ or R′₁, and R₂ or R′₂, R₃ or R′₃ and R₄ or R′₄, R₅ or R′₅ and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and one of R, R′, R₁, R′₁, R₂, R′₂, R₃, R′₃, R₄, R′₄, R₅, R′₅, R₆, R′₆, R₇, R′₇, R₈, R′₈, R₉, and R′₉ together with a different one of R, R′, R₁, R′₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula
—(CH₂)ₓ-M-(CH₂)_{w}-L-(CH₂)_{z}—I—(CH₂)_{y}—
wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and combinations thereof;
and wherein X, Y and Z are independently selected from the group consisting of halide, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins.

56. The method of claim 53, wherein the catalyst is a porphyrin ligand complex or a substituted porphyrin ligand complex.

57. The method of claim 56, wherein the porphyrin ligand complex is selected from the group consisting of manganese (II) porphyrin complexes, manganese(III) porphyrin complexes, iron (II) porphyrin complexes, and iron(III) porphyrin complexes.

58. The method of claim 57 wherein the porphyrin ligand complex is a 5,10,15, 20-tetrakis(2,4,6-trimethyl-3,5-disulfonatophenyl)-porphyrinato iron (III) (FeTMPS).

59. A method for treatment or prophylaxis of hypovolemic shock by inhibiting hypotension in a mammal, said method comprising administering to the mammal a mean arterial pressure sustaining amount of a catalyst for the dismutation of superoxide.

60. The method of claim 59 wherein the catalyst is a non-proteinaceous catalyst, and the non proteinaceous catalyst comprises an organic ligand chelated to a metal ion selected from the group of manganese(II), manganese(II), iron(II) and iron(III).

61. The method of claim 60, wherein the catalyst is a pentaaza-macrocyclic ligand complex.

62. The method of claim 61, wherein the pentaaza-macrocyclic ligand complex is selected from the group consisting of manganese and iron chelates of pentaazacyclopentadecane compounds, which are represented by the following formula: 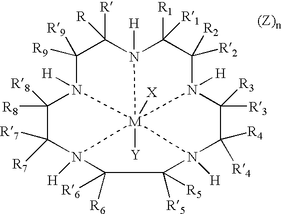
wherein M is a cation of a transition metal, preferably manganese or iron; wherein R, R′, R₁, R′₁, R₂, R′₂, R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R₁ or R′₁ and R₂ or R′₂, R₃ or R′₃ and R₄ or R′₄, R₅ or R′₅ and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and one of R, R′, R₁, R′₁, R₂, R′₂, R₃, R′₃, R₄, R′₄, R₅, R′₅, R₆, R′₆, R₇, R′₇, R₈, R′s, R₉, and R′₉ together with a different one of R, R′, R₁, R′₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula
—(CH₂)ₓ-M-(CH₂)_{w}-L-(CH₂)_{z}—I—(CH₂)_{y}—
wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and combinations thereof;
and wherein X, Y and Z are independently selected from the group consisting of halide, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins.

63. The method of claim 60, wherein the catalyst is a porphyrin ligand complex or a substituted porphyrin ligand complex.

64. The method of claim 63, wherein the porphyrin ligand complex is selected from the group consisting of manganese (II) porphyrin complexes, manganese(III) porphyrin complexes, iron (II) porphyrin complexes, and iron(III) porphyrin complexes.

65. The method of claim 64 wherein the porphyrin ligand complex is a 5,10,15, 20-tetrakis(2,4,6-trimethyl-3,5-disulfonatophenyl)-porphyrinato iron (III) (FeTMPS).

66. A method for treatment or prophylaxis of anaphylactic shock by inhibiting hypotension in a mammal, said method comprising administering to the mammal a mean arterial pressure sustaining amount of a catalyst for the dismutation of superoxide.

67. The method of claim 66 wherein the catalyst is a non-proteinaceous catalyst, and the non proteinaceous catalyst comprises an organic ligand chelated to a metal ion selected from the group of manganese(II), manganese(III), iron(II) and iron(III).

68. The method of claim 67, wherein the catalyst is a pentaaza-macrocyclic ligand complex.

69. The method of claim 68, wherein the pentaaza-macrocyclic ligand complex is selected from the group consisting of manganese and iron chelates of pentaazacyclopentadecane compounds, which are represented by the following formula: 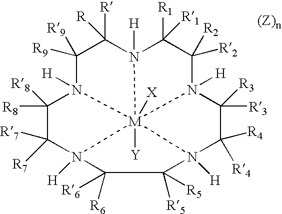
wherein M is a cation of a transition metal, preferably manganese or iron; wherein R, R′, R₁, R′₁, R₂, R′₂, R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R₁ or R′₁, and R₂ or R′₂, R₃ or R′₃ and R₄ or R′₄, R₅ or R′₁, and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and one of R, R′, R₁, R′₁, R₂, R′₂, R₃, R′₃, R₄, R′₄, R₅, R′₅, R₆, R′₆, R₇, R′₇, R₈, R′₈, R₉, and R′₉ together with a different one of R, R′, R₁, R′₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula
—(CH₂)ₓ-M-(CH₂)_{w}-L-(CH₂)_{z}—I—(CH₂)_{y}—
wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and combinations thereof;
and wherein X, Y and Z are independently selected from the group consisting of halide, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins.

70. The method of claim 67, wherein the catalyst is a porphyrin ligand complex or a substituted porphyrin ligand complex.

71. The method of claim 70, wherein the porphyrin ligand complex is selected from the group consisting of manganese (II) porphyrin complexes, manganese(III) porphyrin complexes, iron (II) porphyrin complexes, and iron(III) porphyrin complexes.

72. The method of claim 71 wherein the porphyrin ligand complex is a 5,10,15, 20-tetrakis(2,4,6-trimethyl-3,5-disulfonatophenyl)-porphyrinato iron (III) (FeTMPS).
